(19) United States
(12) Patent Application Publication
(10) Pub. No.: US 2003/0150447 A1
(43) Pub. Date: Aug. 14, 2003

(54) Metered dose inhaler

(76) Inventor: Mark Andrew Hailey, Ware (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/220,486

(22) PCT Filed: Feb. 28, 2001
(86) PCT No.: PCT/EP01/02212

(51) Int. Cl. A61M 11/00

(52) U.S. Cl. 128/200.23

(57) ABSTRACT

There is provided according to the invention a metered dose inhaler comprising an interfacial surface having the following properties: i) a contact angle of greater than 70 degrees; and ii) a conductivity of greater than 2.4 mS. There are also provided processes for its preparation and its use in therapy.

The present invention relates to metered dose inhalers. More especially, the invention relates to a metered dose inhaler for consistently dispensing a prescribed dose of medicament.

Drugs for treating respiratory and nasal disorders are frequently administered in aerosol formulations through the mouth or nose. One widely used method for dispensing such aerosol drug formulations involves formulating the drug as a suspension or a solution in a liquefied gas propellant. The suspension/solution is stored in a sealed canister capable of withstanding the pressure required to maintain the propellant as a liquid. The suspension/solution is dispersed by activation of a dose metering valve affixed to the canister.

A metering valve generally comprises a metering chamber which is of a set volume and is designed to administer per actuation an accurate predetermined dose of medicament. As the suspension is forced from the canister through the dose metering valve by the high vapour pressure of the propellant, the propellant rapidly vaporizes leaving a fast moving cloud of very fine particles of the drug formulation. This cloud of particles is directed into the nose or mouth of the patient by a channeling device such as a cylinder or open-ended cone. Concurrently with the activation of the aerosol dose metering valve, the patient inhales the drug particles into the lungs or nasal cavity. Systems of dispensing drugs in this way are known as “metered dose inhalers” (MDI's). See Peter Byron, Respiratory Drug Delivery, CRC Press, Boca Raton, Fla. (1990) for a general background on this form of therapy.

Patients often rely on medication delivered by MDI's for rapid treatment of respiratory disorders which are debilitating and in some cases even life threatening. Therefore, it is essential that the prescribed dose of aerosol medication delivered to the patient consistently meet the specifications claimed by the manufacturer and comply with the requirements of the FDA and other regulatory authorities. That is, every dose dispensed from the can must be the same within close tolerances.

A problem which can exist with drug delivery devices such as MDI's is the deposition of the medicament, or the solid component from a suspension of a particulate product in a liquid propellant, onto the internal surfaces of the device which occurs after a number of operation cycles and/or storage. This can lead to a reduction in the efficacy of the device and of the resulting treatment as the deposition of the product reduces the amount of active drug available to be dispensed to the patient and markedly reduces the uniformity of the dose dispensed during the lifetime of the device.

The problem of drug adherence and dose uniformity can be greater with hydrofluoroalkane propellants, for example, 1,1,1,2-tetrafluoroethane (HFA134a) and 1,1,1,2,3,3,3-n-heptafluoropropane (HFA227) which have been developed as ozone friendly replacements of chlorofluorocarbons such as P11, P114 and P12.

Some prior art devices rely on the dispenser being shaken so as to agitate the liquid propellant and product mixture therein, in an attempt to dislodge the deposited particles. However, while in some cases this remedy can be effective within the body of the drug container itself, it may not be effective for particles deposited on the inner surfaces of other MDI components such as the metering valve.

UK patent application GB-A-2,328,932 discloses the use of a liner of a material such as fluoropolymer, ceramic or glass to line a portion of the wall of the metering chamber in a metering valve of an MDI. Although this alleviates the problem of deposition in these types of dispensers, it does require the re-design or modification of mouldings and mould tools for producing the valve members to allow for insertion of the liner.

Canadian patent application 2130867 describes a metered dose inhaler containing an aerosol formulation in which the internal walls of the canister are coated with a cross-linked plastics coating. Polytetrafluoroethylene (PTFE) and perfluoroethylenepropylene (FEP) are specifically mentioned as suitable coating materials. International patent application PCT/US96/05005 (WO96/32150) describes a metered dose inhaler in which part or all of the internal surfaces of the canister are coated with a cross-linked polymeric composition, particularly polymer blends comprising one or more fluorocarbon polymers in combination with one or more non-fluorocarbon polymers.

Whilst the aforementioned polymer coatings minimize deposition of the drug onto the walls of the canister or other MDI components, certain technical disadvantages are associated with this approach. For example, the component may deform as a result of being subject to the elevated temperatures, typically in excess of 300° C., required for the coating process. Therefore, components have to be formed from thicker sheets of material which increases costs and the quantity of waste material. Furthermore, difficulties arise in ensuring adhesion of the polymer to the component walls and more particularly with uniformity of the coating over the component surface.

Perhaps most importantly, it has been found that the use of such polymer coatings can under certain circumstances lead to a variation in the uniformity of dose from first use through to the emptying of the MDI device.

Unexpectedly, the present inventors have found that by providing the components as described herein, the need to apply a cross-linked polymer coating material, and therefore the disadvantages associated therewith, is obviated. MDI's and components thereof so provided, advantageously reduce drug deposition onto the walls of the components and afford greater dose uniformity over the lifetime of the device.

Accordingly, in one aspect the invention provides a metered dose inhaler comprising an interfacial surface having the following properties:

(i) a contact angle of greater than 70 degrees; and

(ii) a conductivity of greater than 2.4 mS.

In another aspect, the invention provides a component or accessory for use in a metered dose inhaler comprising an interfacial surface having the following properties:

(i) a contact angle of greater than 70 degrees; and

(ii) a conductivity of greater than 2.4 mS.

As used herein, the term “interfacial surface” defines all or part of any internal surface of the metered dose inhaler, component or accessory, that contacts or comes into contact, i.e. forms an interface with, a medicament during storage and/or dispensing thereof.

As used herein, the term “metered dose inhaler” or “MDI” means a unit comprising a canister, a cap covering the mouth of the canister, a drug metering valve situated in the cap, a metering chamber and a suitable channeling device into which the canister is fitted. The term “drug metering valve” or “MDI valve” refers to a valve and its associated mechanisms which delivers a predetermined amount of drug formulation from an MDI upon each activation. The channeling device may comprise, for example, an actuating device for the valve and a cylindrical or cone-like passage through which medicament may be delivered from the filled MDI can via the MDI valve to the nose or mouth of a patient, e.g. a mouthpiece actuator. The relation of the parts of a typical MDI is illustrated in U.S. Pat. No. 5,261,538 incorporated herein by reference.

Therefore, the component or accessory may include a canister, and/or a metering valve, and/or a metering chamber, and/or a channeling device and/or an actuator for use in a metered dose inhaler.

Preferably, the contact angle of the interfacial surface is greater than 90 degrees, e.g. greater than 110 degrees.

As used herein, “contact angle” is identified as the angle between a liquid water droplet and a solid surface at the liquid/solid gas interface.

Preferably, the conductivity of the interfacial surface is greater than 4.0 mS. Typically, the conductivity is greater than 7.9 mS.

As used herein, “conductivity” is evaluated by applying a low voltage of 6.3V between the surface and a salt (e.g. 1% sodium chloride) solution alongside the surface, using a WACO™ Enamel Rater II Balance, i.e. using the WACO Conductivity Test for the Determination of Coating Integrity of Metered Dose Inhalers. Therefore, measurements from products according to the invention according to this apparatus are greater than 15 mA, typically greater than 25 mA, e.g. greater than 50 mA, which corresponds to a conductivity of greater than 2.4 mS, 4.0 mS and 7.9 mS respectively.

The products according to the present invention reduce the variation in dosage with respect to a conventionally polymer coated metered dose inhaler.

Preferably, the metered dose inhaler is suitable for consistently dispensing a dose of medicament ranging between 90 and 110% of a prescribed single dosage. Typically, the metered dose inhaler is suitable for dispensing a dose of medicament ranging, for example, between 95 and 105% of a prescribed single dosage, e.g. between 97% and 103%, such as between 98% and 102%, of a prescribed single dosage.

Mean dose is calculated by taking ten metered dose inhalers. The beginning of use (BOU) dose and the end of use (EOU) dose is measured for each of the ten inhalers. The mean of the 20 measurements is then calculated. The dosing consistency is calculated by looking at the dose from BOU to EOU and quoting the mean result from each of the 10 determinations as a percentage of the overall mean.

As used herein, “consistently dispensing” defines the dose uniformity of the aerosol medication dispensed to the patient from the first dose through to the final dose dispensed from the drug canister in the MDI device.

In one embodiment, the interfacial surface may have a linear, non-cross-linked polymeric compound disposed thereon.

Typically, the polymeric compound is disposed as a multi-molecular layer thereon. Alternatively, the polymeric compound is disposed as a mono-molecular layer thereon.

Preferably, the polymeric compound is a fluorocarbon. In particular, the fluorocarbon is highly fluorinated, e.g. has a high fluorine to carbon ratio.

The polymeric compounds will generally be employed as mixtures, the nature of which may be varied as part of optimisation of the employment of the invention.

Preferably, the polymeric compound comprises a functional grouping which is capable of anchoring the compound to the surface of the substrate (e.g. component).

For example, in a first embodiment the compound may be an organo-phosphate such as a phosphate based perfluoroether derivative. Typically, the compound is a phosphoric ester.

In one first such embodiment, the interfacial surface has a compound disposed thereon having the general formula (I):

R¹—(OC₃F₆)ₓ—(OCF₂)_{y}—R² (I)

wherein R¹ comprises a fluoro-alkyl functional group;

x and y are such that the molecular weight of the compound is in the range 350-1000; and

R² comprises a phosphoric ester functional group.

In a second such embodiment, the interfacial surface has a compound disposed thereon having the general formula (II):

R³—(CH₂)ᵥ—CF₂O—(C₂F₄O)ₓ—(CF₂O)_{y}CF₂—(CH₂)_{w}—R³ (II)

wherein R³ comprises —(OCH₂—CH₂)_{z}—OPO(OH)₂;

x, y and z are such that the molecular weight of the compound is in the range 900-2100; and

v and w independently represent 1 or 2.

In one preferred embodiment, v and w are both 1. In a second preferred embodiment v and w are both 2.

Alternatively in a second embodiment the compounds may be an organo-silane derivative such as a silane derivative of perfluoropolyoxyalkane, e.g. a silane derivative of perfluoropolyoxyalkane having a molecular weight in the range 1600-1750. Examples include perfluoropolyoxyalkanes having functional groups of the type—CONR⁴R⁵ wherein R⁴ and R⁵ may be independently selected from hydrogen, or a silyl ether (e.g. SiRₜ(OR)₃₋ₜ) wherein R=hydrogen or C₁₋₈alkyl and t=0 to 2) as described in U.S. Pat. No. 4,746,550 which is incorporated herein by reference.

The synthesis of compounds of formula (I) and (II) may readily be determined by reference to EP 687 533 which describes similar compounds. EP 338 531 also provides information on the preparation of compounds of this type. Methods of preparing organo-silane polymeric compounds of the type described above may readily be determined by reference to U.S. Pat. No. 4,746,550.

Whilst not wishing to be bound by any theory, it is believed that the phosphate or silane moiety of the compounds of formula as described above reacts with the surface of the component to anchor the compound to the surface. Thus, when in use, the per-fluorinated end of the compound is presented to the pharmaceutical formulation and so provides a highly fluorinated surface.

The interfacial surface may be a metallic or plastics surface. Preferably, the interfacial surface is metallic.

In a first preferred embodiment, the component or accessory having an interfacial surface according to the invention is a canister. In a second preferred embodiment the component or accessory having an interfacial surface according to the invention is a metering valve, especially a metering chamber.

In another embodiment, the metered dose inhaler or a component or accessory is suitable for storing and/or dispensing a medicament, and deposition of the medicament on the MDI, component or accessory during storage and/or dispensing may be reduced by between 30% and 80%, for example, between 40% and 80%, e.g. between 40% and 60%, such as about 50%.

As used herein, the reference to the reduction in deposition of medicament is with respect to the deposition that would occur on a metered dose inhaler, component or accessory which does not comprise an interfacial surface having the properties defined above.

In a further aspect, there is provided a canister as described above for use in a metered dose inhaler, containing a pharmaceutical aerosol formulation comprising a medicament and a fluorocarbon propellant.

Aerosol formulations which are generally used comprise a suspension of medicament, one or more liquid propellants, optionally with co-propellants and optionally an adjuvant or a surfactant, though the invention may be applicable to the dispensing of any aerosol formulation.

In another aspect of the invention there is provided a metered dose inhaler comprising a canister, and/or a metering valve, and/or a metering chamber, and/or a channeling device and/or an actuator as described above.

In still another aspect, the invention provides the use of a metered dose inhaler, component or accessory as described above, for dispensing a pharmaceutical aerosol formulation comprising a medicament and a fluorocarbon propellant.

In yet a further aspect, the invention provides a process for obtaining a metered dose inhaler, or a component or accessory for use in a metered dose inhaler as described above, comprising the treatment of an interfacial surface thereof with a linear, non-cross-linked polymeric compound.

Preferably, the interfacial surface is treated to form a multi-molecular layer thereon, which may be applied as separate layers wherein the layers need not be the same polymeric compound. More preferably, the interfacial surface is treated to form amono-molecular layer thereon.

In a preferred embodiment, the polymeric compound is a fluorocarbon. Typically, the compound is highly fluorinated.

Typically, the linear, non-cross-linked polymeric compound comprises a functional grouping which is capable of anchoring the compound to the surface to be treated. Preferably, the compound is an organo-phosphate, for example, a phosphate based perfluoroether derivative or a silane derived perfluorpolyoxyalkane. Typically, the compound takes the form of a phosphoric ester or a silane derivative of perfluoropolyoxyalkanes having a molecular weight in the range 1600-1750.

The inventors have found that such treatment of MDIs or one or more components thereof results in an increase in the uniformity of the dose dispensed with dose number through to the emptying of the drug canister. Advantageously, unlike the use of polymer linings or coatings, the present process does not require the re-design or modification of mouldings and mould tools for producing the valve members to allow for insertion of a liner, or the need to use thick component walls in order to avoid deformation as a result of being subject to elevated temperatures, typically in excess of 300° C., which are required for the coating process. Therefore, components may now be formed from thinner sheets of material which reduces costs and the quantity of waste material. Low temperature treatment also reduces process costs.

Furthermore, difficulties arising in ensuring adhesion of a polymer to the component walls and more particularly with uniformity of the coating over the component surface are obviated. Preferably, the process for obtaining a metered dose inhaler, component or accessory as defined above, may comprises the treatment of an interfacial surface with a compound:

i) having the general formula (I)

R¹—(OC₃F₆)ₓ—(OCF₂)_{y}—R² (I)

wherein R¹ comprises a fluoro-alkyl functional group;

x and y are such that the molecular weight of the compound is 350-1000; and

R² comprises a phosphate ester functional group; or

ii) having a general formula (II)

R³—(CH₂)ᵥ—CF₂O—(C₂F₄O)ₓ—(CF₂O)_{y}CF₂—(CH₂)_{w}—R³ (II)

wherein R³ comprises —(OCH₂—CH₂)_{z}—OPO(OH)₂;

x, y and z are such that the molecular weight of the compound is 900-2100; and

v and w independently represent 1 or 2; or

iii) a silane derivative of perfluoropolyoxyalkane with a molecular weight in the range 1600-1750.

The inventors also contemplate that the manufacturing machinery used to produce MDI's, their components and accessories may also comprise the properties defined in accordance with the invention. Furthermore, apparatus for filling empty canisters, or other MDI components, with medicament may also comprise such properties. In this way, inaccuracies due to deposition or drug metering may be prevented at the stage of loading the MDI with its full load of medicament.

The metered dose inhalers may be prepared by methods of the art (e.g. see Byron above and U.S. Pat. No. 5,345,980) substituting conventional canisters for those treated in accordance with the present invention.

Conventionally, the canisters and caps for use in MDI's are made of aluminium or an alloy of aluminium although other metals not affected by the drug formulation, such as stainless steel, an alloy of copper, or tin plate, may be used. An MDI canister may also be fabricated from glass or plastic. Preferably, however, the MDI canisters and caps employed in the present invention are made of aluminium or an alloy thereof.

The drug metering valve may consist of parts usually made of stainless steel, a pharmacologically resilient polymer, such as acetal, polyamide (e.g. Nylon^{R}), polycarbonate, polyester, fluorocarbon polymer (e.g. Teflon^{R}) or a combination of these materials. Additionally, seals and “O” rings of various materials (e.g., nitrile rubbers, polyurethane, acetyl resin, fluorocarbon polymers), or other elastomeric materials are employed in and around the valve.

The components of the MDI described hereinabove may be pretreated as coil stock, such as aluminium or stainless steel, before being stamped or drawn into shape. This method is well suited to high volume production due to the high standards of uniformity that can be achieved and to the high speed and precision with which pre-coated stock can be drawn or stamped.

Alternatively, the components may be manufactured according to a second process comprising treating pre-formed canisters.

Preferably, the components or coil stock are dipped or bath immersed into a treatment tank containing a solution the polymeric compound as described above or a mixture thereof.

The components or coil stock may be treated with 0.1 to 10% w/w, preferably 0.5 to 5%, especially about 1%, solution of a polymeric compound as described above or a mixture thereof in any suitable solvent such as isopropyl alcohol.

Conventional metal coating techniques such as spraying and immersion may be used to apply the treatment solution to the pre-formed components or coil stock. Preferably, the preformed components or the coil stock are immersed in the solution at room temperature for at least one hour, for example, 12 hours, thus being treated both internally and externally.

The treatment solution may also be poured inside the MDI components then drained to treat the internal component (e.g. the inner surface of a canister) only.

The treated canisters are preferably washed with solvent and dried at an elevated temperature for example 50-100° C. optionally under vacuum.

In medical use the canisters in accordance with the invention contain a pharmaceutical aerosol formulation comprising a medicament and a fluorocarbon or hydrogen-containing chlorofluorocarbon propellant.

Suitable propellants include, for example, C₁₋₄hydrogen-containing chlorofluorocarbons such as CH₂ClF, CClF₂CHClF, CF₃CHClF, CHF₂CClF₂, CHClFCHF₂, CF₃CH₂Cl and CClF₂CH₃; C₁₋₄hydrogen-containing fluorocarbons such as CHF₂CHF₂, CF₃CH₂F, CHF₂CH₃ and CF₃CHFCF₃; and perfluorocarbons such as CF₃CF₃ and CF₃CF₂CF₃.

Where mixtures of the fluorocarbons or hydrogen-containing chlorofluorocarbons are employed they may be mixtures of the above identified compounds or mixtures, preferably binary mixtures, with other fluorocarbons or hydrogen-containing chlorofluorocarbons for example CHClF₂, CH₂F₂ and CF₃CH₃. Preferably a single fluorocarbon or hydrogen-containing chlorofluorocarbon is employed as the propellant. Particularly preferred as propellants are C₁₋₄hydrogen-containing fluorocarbons such as 1,1,1,2-tetrafluoroethane (CF₃CH₂F) and 1,1,1,2,3,3,3-heptafluoro-n-propane (CF₃CHFCF₃) or mixtures thereof. 1,1,1,2-Tetrafluoroethane is of particular interest.

Preferably the pharmaceutical formulations for use in the canisters of the invention contain no components which provoke the degradation of stratospheric ozone. In particular the formulations are substantially free of chlorofluorocarbons such as CCl₃F, CCl₂F₂ and CF₃CCl₃.

The propellant may additionally contain a volatile adjuvant such as a saturated hydrocarbon for example propane, n-butane, isobutane, pentane and isopentane or a dialkyl ether for example dimethyl ether. In general, up to 50% w/w of the propellant may comprise a volatile hydrocarbon, for example 1 to 30% w/w. However, formulations which are free or substantially free of volatile adjuvants are preferred. In certain cases, it may be desirable to include appropriate amounts of water, which can be advantageous in modifying the dielectric properties of the propellant.

A polar co-solvent such as C₂₋₆ aliphatic alcohols and polyols e.g. ethanol, isopropanol and propylene glycol, preferably ethanol, may be included in the drug formulation in the desired amount to improve the dispersion of the formulation, either as the only excipient or in addition to other excipients such as surfactants. Suitably, the drug formulation-may contain 0.01 to 5% w/w based on the propellant of a polar co-solvent e.g. ethanol, preferably 0.1 to 5% w/w e.g. about 0.1 to 1% w/w.

A surfactant may also be employed in the aerosol formulation. Examples of conventional surfactants are disclosed in EP 372 777 incorporated herein by reference. The amount of surfactant employed is desirable in the range 0.0001% to 50% weight to weight ratio relative to the medicament, in particular, 0.05 to 5% weight to weight ratio. Preferred surfactants are lecithin, oleic acid and sorbitan trioleate. Preferred formulations, however, are free or substantially free of surfactant.

Pharmaceutical formulations may contain 0.0001 to 50% w/w, preferably 0.001 to 20%, for example 0.001 to 1% of sugar relative to the total weight of the formulation. Generally the ratio of medicament to sugar falls within the range of 1:0.01 to 1:100 preferably 1:0.1 to 1:10. Typical sugars which may be used in the formulations include, for example, sucrose, lactose and dextrose, preferably lactose, and reducing sugars such as mannitol and sorbitol, and may be in micronised or milled form.

The final aerosol formulation desirably contains 0.005-10% w/w, preferably 0.005 to 5% w/w, especially 0.01 to 1.0% w/w, of medicament relative to the total weight of the formulation.

Medicaments which may be administered in aerosol formulations according to the invention include any drug useful in inhalation therapy and which may be presented in a form which is substantially completely insoluble in the selected propellant. Appropriate medicaments may thus be selected from, for example, analgesics, e.g. codeine, dihydromorphine, ergotamine, fentanyl or morphine; anginal preparations, e.g. diltiazem; anti-allergics, e.g. cromoglycate (e.g. as sodium salt), ketotifen or nedocromil (e.g. as sodium salt); antiinfectives e.g. cephalosporins, penicillins, streptomycin, sulphonamides, tetracyclines and pentamidine; anti-histamines, e.g. methapyrilene; anti-inflammatories, e.g. beclomethasone (e.g. as dipropionate), fluticasone (e.g. as propionate), flunisolide, budesonide, rofleponide, mometasone (e.g. as furoate), ciclesonide, triamcinolone (e.g. as acetonide) or 6α, 9α-difluoro-11β-hydroxy-16α-methyl-3-oxo-17α-propionyloxy-androsta-1,4-diene-17β-carbo-thioic acid S-(2-oxo-tetrahydro-furan-3-yl) ester; anti-tussives, e.g. noscapine; bronchodilators, e.g. albuterol (e.g. as free base or as sulphate), salmeterol (e.g. as xinafoate), ephedrine, adrenaline, fenoterol (e.g. as hydrobromide), formoterol (e.g. as fumarate), isoprenaline, metaproterenol, phenylephrine, phenylpropanolamine, pirbuterol (e.g. as acetate), reproterol (e.g. as hydrochloride), rimiterol, terbutaline (e.g. as sulphate), isoetharine, tulobuterol, 4-hydroxy-7-[2-[[2-[[3-(2-phenylethoxy)propyl]sulfonyl]ethyl]amino]-ethyl-2(3H)-benzothia-zolone; diuretics, e.g. amiloride; anti-cholinergics, e.g. ipratropium (e.g. as bromide), tiotropium, atropine or oxitropium; hormones, e.g., cortisone, hydrocortisone or prednisolone; xanthines, e.g. aminophylline, choline theophyllinate, lysine theophyllinate or theophylline. It will be clear to a person skilled in the art that, where appropriate, the medicaments may be used in the form of salts, (e.g. as alkali metal or amine salts or as acid addition salts) or as esters (e.g. lower alkyl esters) or as solvates (e.g., hydrates) to optimise the activity and/or stability of the medicament and/or to minimise the solubility of the medicament in the propellant. It will further be clear to a person skilled in the art that where appropriate, the medicaments may be used in the form of a pure isomer, for example, R-albuterol or RR-formoterol.

Particularly preferred medicaments for administration using aerosol formulations in accordance with the invention include anti-allergics, bronchodilators and anti-inflammatory steroids of use in the treatment of respiratory disorders such as asthma by inhalation therapy, for example cromoglycate (e.g. as the sodium salt), salbutamol (e.g. as the free base or the sulphate salt), salmeterol (e.g. as the xinafoate salt), formoterol (e.g. as the fumarate salt), terbutaline (e.g. as the sulphate salt), reproterol (e.g. as the hydrochloride salt), a beclomethasone ester (e.g. the diproprionate), a fluticasone ester (e.g. the propionate). Salmeterol, especially salmeterol xinafoate, salbutamol, fluticasone propionate, beclomethasone dipropionate and physiologically acceptable salts and solvates thereof are especially preferred.

It will be appreciated by those skilled in the art that the aerosol formulations according to the invention may, if desired, contain a combination of two or more active ingredients. Aerosol compositions containing two active ingredients are known for the treatment of respiratory disorders such as asthma, for example, formoterol and budesonide, salmeterol (e.g. as the xinafoate salt) and fluticasone (e.g. as the propionate ester), salbutamol (e.g as the free base or sulphate) and beclomethasone (as the dipropionate ester) are preferred.

Particularly preferred is a combination of fluticasone propionate and salmeterol, or a salt thereof (particularly the xinafoate salt).

Particularly preferred formulations for use in the canisters of the present invention comprise a medicament and a C₁₋₄ hydrofluoroalkane particularly 1,1,1,2-tetrafluoroethane and 1,1,1,2,3,3,3-n-heptafluoropropane or a mixture thereof as propellant, especially 1,1,1,2-tetrafluoroethane.

Preferred formulations are free or substantially free of formulation excipients. Thus, preferred formulations consist essentially of (or consist of) the medicament and the selected propellant.

Conventional bulk manufacturing methods and machinery well known to those skilled in the art of pharmaceutical aerosol manufacture may be employed for the preparation of large scale batches for the commercial production of filled canisters. Thus, for example, in one bulk manufacturing method a metering valve is crimped onto an aluminium can to form an empty canister. The particulate medicament is added to a charge vessel and liquified propellant is pressure filled through the charge vessel into a manufacturing vessel. The drug suspension is mixed before re-circulation to a filling machine and an aliquot of the drug suspension is then filled through the metering valve into the canister.

In an alternative process, an aliquot of the liquefied formulation is added to an open canister under conditions which are sufficiently cold such that the formulation does not vaporise, and then a metering valve crimped onto the canister.

Preferably the canister is fitted with a cap assembly, wherein a formulation metering valve is situated in the cap, and said cap is crimped in place. The cap may be secured onto the canister via welding such as ultrasonic welding or laser welding, screw fitting or crimping.

MDIs taught herein may be prepared by methods of the art (e.g., see Byron, above and WO/96/32150) substituting conventional cans for those treated in accordance with the present invention.

Typically, in batches prepared for pharmaceutical use, each filled canister is check-weighed, coded with a batch number and packed into a tray for storage before release testing.

Each filled canister is conveniently fitted into a suitable channeling device prior to use to form a metered dose inhaler for administration of the medicament into the lungs or nasal cavity of a patient. Suitable channeling devices comprise for example a valve actuator and a cylindrical or cone-like passage through which medicament may be delivered from the filled canister via the metering valve to the nose or mouth of a patient e.g. a mouthpiece actuator. Metered dose inhalers are designed to deliver a fixed unit dosage of medicament per actuation or “puff”, for example in the range of 10 to 5000 microgram medicament per puff.

Administration of medicament may be indicated for the treatment of mild, moderate or severe acute or chronic symptoms or for prophylactic treatment. It will be appreciated that the precise dose administered will depend on the age and condition of the patient, the particular particulate medicament used and the frequency of administration and will ultimately be at the discretion of the attendant physician. When combinations of medicaments are employed the dose of each component of the combination will in general be that employed for each component when used alone. Typically, administration may be one or more times, for example from 1 to 8 times per day, giving for example 1,2,3 or 4 puffs each time. Each valve actuation, for example, may deliver 5 μg, 50 μg, 100 μg, 200 μg or 250 μg of a medicament. Typically, each filled canister for use in a metered dose inhaler contains 60, 100, 120 or 200 metered doses or puffs of medicament; the dosage of each medicament is either known or readily ascertainable by those skilled in the art.

A still further aspect of the present invention comprises a method of treating respiratory disorders such as, for example, asthma, which comprises administration by inhalation of an effective amount of an aerosol formulation as herein described from a metered dose inhaler of the present invention.

### EXAMPLES

### Example 1

Standard 12.5 ml MDI canisters (Presspart Inc Cary N.C.) are immersed in a solution of 1% w/w compound of formula (I) in isopropyl alcohol for 12 hours at room temperature. The canisters are then drained and allowed to dry at 80° C. under vacuum. The cans are then purged of air and the valves crimped in place, and a suspension of about 31.8 mgsalbutamol sulphate in about 19.8 g HFA 134a is filled through the valve.

### Example 2

Example 1 is repeated except a suspension of about 4.25 mg salmeterol xinafoate and about 8 g HFA 134a is filled through the valve.

### Example 3

Example 1 is repeated except a suspension of 22 mg fluticasone propionate and 15 g HFA 134a is filled through the valve.

### Example 4

Example 1 is repeated except a suspension of about 44 mg fluticasone propionate and about 12 g HFA 134a is filled through the valve.

### Example 5

Example 1 is repeated except a suspension of about 13.8 mg fluticasone propionate with about 4 mg salmeterol xinafoate and 8 g HFA 134a is filled through the valve.

### Example 6

Example 1 is repeated except a suspension of about 29 mg fluticasone propionate with about 21.4 g HFA 227 is filled through the valve.

### Example 7-12

Examples 1 to 6 are repeated except that a compound of formula (II) is employed instead of a compound of formula (I).

### Examples 13-18

Examples 1 to 6 are repeated except that a silane derivative of perfluoropolyoxyalkane with a molecular weight in the range 1600-1750 is employed instead of a compound of formula (I).

It will be understood that the present disclosure is for the purpose of illustration only and the invention extends to modifications, variations and improvements thereto which will be within the ordinary skill of the person skilled in the art.

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

1. A component or accessory for use in a metered dose inhaler comprising an interfacial surface having the following properties:—
(i)a contact angle of greater than 70 degrees; and
(ii) a conductivity of greater than 2.4 mS.
wherein the interfacial surface has a linear, non-cross-linked polymeric compound disposed thereon and said polymeric compound comprises a functional grouping which is capable of anchoring the said compound to the said surface.

2. A metered dose inhaler comprising a component or accessory as claimed in claim 1.

3. A component or accessory as claimed in claim 2 selected from the group consisting of a canister, a metering valve, a metering chamber, a channeling device and an actuator for use in a metered dose inhaler.

4. A metered dose inhaler as claimed in claim 1 or a component or accessory for use in a metered dose inhaler as claimed in claim 2 or claim 3 wherein the contact angle of the interfacial surface is greater than 90 degrees.

5. A metered dose inhaler as claimed in claim 1 or claim 4, or a component or accessory as claimed in claim 2 to 5, wherein the conductivity of the interfacial surface is greater than 4.0 mS.

6. A metered dose inhaler as claimed in any one of claims 1, 4 or 5, or a component or accessory as claimed in any one of claims 2 to 5, wherein the metered dose inhaler is suitable for consistently dispensing a dose of a medicament ranging between 90 and 110% of a prescribed single dosage.

7. A metered dose inhaler, or a component or accessory as claimed in any one of claims 1 or 4 to 6 wherein the polymeric compound is disposed as a multi-molecular layer thereon.

8. A metered dose inhaler, or a component or accessory as claimed in any one of claims 1 or 4 to 6 wherein the polymeric compound is disposed as a mono-molecular layer thereon.

9. A metered dose inhaler, or a component or accessory as claimed in any one of claims 7 or 8 wherein the polymeric compound is a fluorocarbon.

10. A metered dose inhaler, a component or accessory as claimed in claim 9 wherein the fluorocarbon is highly fluorinated.

11. A metered dose inhaler, component or accessory as claimed in any one of claims 2 to 10 wherein the compound is an organo-phosphate.

12. A metered dose inhaler, component or accessory as claimed in claim 11 wherein the compound is a phosphate based perfluoroether derivative.

13. A metered dose inhaler, component or accessory as claimed in claim 11 or claim 12 wherein the compound is a phosphoric ester.

14. A metered dose inhaler, component or accessory as claimed in claim 13 wherein the interfacial surface has a compound disposed thereon having the general formula (I):
R¹—(OC₃F₆)ₓ—(OCF₂)_{y}—R² (I)
wherein R¹ comprises a fluoro-alkyl functional group;
x and y are such that the molecular weight of the compound is 350-1000; and
R² comprises a phosphoric ester functional group.

15. A metered dose inhaler, component or accessory as claimed in claim 13 wherein the interfacial surface has a component disposed thereon having the general formula (II):
R³—(CH₂)ᵥ—CF₂O—(C₂F₄O)ₓ—(CF₂O)_{y}CF₂—(CH₂)_{w}—R³ (II)
wherein R³ comprises —(OCH₂—CH₂)_{z}—OPO(OH)₂;
x, y and z are such that the molecular weight of the compound is 900-2100; and
v and w independently represent 1 or 2.

16. A metered dose inhaler, component or accessory as claimed in claim 15 wherein v and w are both 1.

17. A metered dose inhaler, component or accessory as claimed in claim 15 wherein v and w are both 2.

18. A metered dose inhaler, component or accessory as claimed in any one of claims 2 to 10 wherein the compound is an organo-silane derivative.

19. A metered dose inhaler, component or accessory as claimed in claim 18, wherein the compound is a silane derivative of a perfluoropolyoxyalkane.

20. A metered dose inhaler, component or accessory as claimed in claim 19, wherein the interfacial surface has a compound disposed thereon which is a silane derivative of a perfluoropolyoxyalkane having a molecular weight in the range 1600-1750.

21. A metered dose inhaler as claimed in any one of claims 2, or 4 to 20, or a component or accessory as claimed in any one of claims 1 to 20, wherein the interfacial surface is a metallic, metal alloy or plastics surface.

22. A metered dose inhaler, component or accessory as claimed in claim 21 wherein the interfacial surface is a metallic or metal alloy surface.

23. A metered dose inhaler as claimed in any one of claims 2, or 4 to 22, or a component or accessory as claimed in any one of claims 1 to 22, for storing and/or dispensing a medicament, wherein, during storage and/or dispensing, deposition of the medicament on the MDI, component or accessory is reduced by between 30% and 80%.

24. A component or accessory as claimed in any one of claims 1 to 23 which comprises a canister containing a pharmaceutical aerosol formulation comprising a medicament, a fluorocarbon propellant and optionally a solvent.

25. A metered dose inhaler comprising a component or accessory as claimed in any one of claims 1 to 24 comprising a canister, and/or a metering valve, and/or a metering chamber, and/or a channeling device and/or an actuator.

26. Use of a metered dose inhaler as claimed in claims 2, 4 to 23 or 25, or a component or accessory as claimed in any one of claims 2 to 25, for dispensing a pharmaceutical aerosol formulation comprising a medicament and a fluorocarbon propellant.

27. Use as claimed in claim 26 wherein the pharmaceutical aerosol formulation to be dispensed is a medicament suspended in propellants selected from liquefied HFA 134a, 227 or a mixture thereof.

28. Use as claimed in claim 26 or 27 wherein the propellant is substantially free of adjuvants.

29. Use as claimed in any one of claims 26 to 28 in which the medicament is selected from fluticasone propionate, salbutamol, beclomethasone dipropionate, salmeterol, pharmaceutically acceptable salts, solvates or esters thereof and mixtures thereof.

30. A process for obtaining a metered dose inhaler as claimed in claims 2, 4 to 23 or 25, or a component or accessory as claimed in any one of claims 1 to 24, comprising the treatment of the interfacial surface with a linear, non-cross-linked polymeric compound.

31. A process as claimed in claim 30 wherein the polymeric compound is disposed as a multi-molecular layer thereon.

32. A process as claimed in claim 30 wherein the polymeric compound is disposed as a mono-molecular layer thereon.

33. A process as claimed in any one of claims 30 to 32 wherein the linear polymeric compound is a fluorocarbon.

34. A process as claimed in claim 33 wherein the fluorocarbon is highly fluorinated.

35. A process as claimed in any one of claims 30 to 34 wherein the polymeric compound comprises a functional grouping which is capable of anchoring the compound to the surface thereof.

36. A process as claimed in claim 35 wherein the compound is an organo-phosphate.

37. A process as claimed in claim 36 wherein the compound is a phosphate-based perfluoroether derivative.

38. A process as claimed in claim 36 or claim 37 wherein the compound is a phosphoric ester.

39. A process as claimed in any one of claims 30 to 35, comprising the treatment of the interfacial surface with a compound having the general formula (I):
R¹—(OC₃F₆)ₓ—(OCF₂)_{y}—R² (I)
wherein R¹ comprises a fluoro-alkyl functional group;
x and y are such that the molecular weight of the compound is 350-1000; and
R² comprises a phosphate ester functional group.

40. A process as claimed in any one of claims 30 to 35 comprising the treatment of the interfacial surface with a compound having the general formula (II)
R³—(CH₂)ᵥ—CF₂O—(C₂F₄O)ₓ—(CF₂O)_{y}CF₂—(CH₂)_{w}—R³ (II)
wherein R³ comprises —(OCH₂—CH₂)_{Z}—OPO(OH)₂;
x, y and z are such that the molecular weight of the compound is 900-2100; and
v and w independently represent 1 or 2.

41. A process as claimed in any one of claims 30 to 35 wherein the compound is an organo-silane derivative.

42. A process as claimed in claim 41 wherein the compound is a silane derivative of perfluoropolyoxyalkane

43. A process as claimed in claim 42 comprising the treatment of the interfacial surface with a silane derivative of perfluoropolyoxyalkane with a molecular weight in the range 1600-1750.
